(19) United States
(12) Patent Application Publication
(10) Pub. No.: US 2006/0046266 A1
(43) Pub. Date: Mar. 2, 2006

(54) Inkjet-type DNA arrayer able to perform PCR and method of manufacturing DNA microarrays using the same

(76) Inventors: Keon Kuk, Gyeonggi-do (KR); Min-soo Kim, Seoul (KR)

Correspondence Address: CANTOR COLBURN, LLP, 55 GRIFFIN ROAD SOUTH, BLOOMFIELD, CT, 06002, US

(21) Appl. No.: 11/216,446

(22) Filed: Aug. 31, 2005

(30) Foreign Application Priority Data
Aug. 31, 2004 (KR) 10-2004-0069089

(51) Int. Cl. C12Q 1/68; C12P 19/34; C12M 1/34; B41J 2/01

(52) U.S. Cl. 435/6; 435/91.2; 435/287.2; 347/1

(57) ABSTRACT

An inkjet-type DNA arrayer for performing PCR and a method of manufacturing DNA microarrays using the same are provided. The inkjet-type DNA arrayer includes: a substrate having a plurality of flow paths filled with a PCR solution; a plurality of first heaters heating the PCR solution according to the PCR temperature cycle; and a plurality of second heaters heating a product solution generated by the PCR to a predetermined temperature to discharge the product solution to outside.

### BACKGROUND OF THE INVENTION

This application claims the benefit of Korean Patent Application No. 10-2004-0069089, filed on Aug. 31, 2004, in the Korean Intellectual Property Office, the disclosure of which is incorporated herein in its entirety by reference.

1. Field of the Invention

The present invention relates to a DNA arrayer and a method of manufacturing DNA microarrays using the same, and more particularly, to a thermal inkjet-type DNA arrayer able to perform a polymerase chain reaction (PCR) and a method of manufacturing DNA microarrays using the same.

2. Description of the Related Art

A polymerase chain reaction (PCR) is typically used to proliferate DNA to an amount sufficient for analysis from a small amount of a sample. The PCR proliferates DNA by repeating a thermal cycling process including denaturation, annealing, and polymerization.

Conventionally, a PCR apparatus that conducts the PCR in a tube using 50-100 μl of a PCR solution was used. However, a micro PCR apparatus that proliferates DNA using about 1 μl of a PCR solution has recently been used. The micro PCR apparatus can continuously perform the PCR and can achieve an automated proliferation process.

FIG. 1 schematically illustrates a cross-section of a general micro PCR apparatus. Referring to FIG. 1, the micro PCR apparatus includes a first substrate 10 and a second substrate 20 attached to each other, and a heater 30 installed directly below and attached to the second substrate 20. A reaction chamber 25 filled with a PCR solution is formed between the first substrate 10 and the second substrate 20.

FIG. 2 is a graph illustrating the temperature distribution of the PCR solution filled in the reaction chamber 25 with respect to time when heating the PCR solution with the heater 30 in the micro PCR apparatus illustrated in FIG. 1. A glass substrate and a silicon substrate are used as the first substrate 10 and the second substrate 20, respectively, and the reaction chamber 25 has a height of 100 μm. In FIG. 2, a lower portion, a middle portion, and an upper portion represent points of 5 μm, 55 μm, and 95 μm, respectively, from the bottom of the reaction chamber 25. Referring to FIG. 2, in the conventional micro PCR apparatus, the temperature distribution of the PCR solution is not uniform at said three portions. When the temperature distribution of the PCR solution is not uniform, the reproducibility of the PCR proliferation result is deteriorated.

### SUMMARY OF THE INVENTION

The present invention provides a thermal inkjet-type DNA arrayer that can simultaneously implement a PCR for DNA proliferation and DNA spotting for manufacturing DNA microarrays, and a method of manufacturing DNA microarrays using the same.

According to an aspect of the present invention, there is provided an inkjet-type DNA arrayer including: a substrate having a plurality of flow paths filled with a PCR solution; a plurality of first heaters to heat the PCR solution according to the PCR temperature cycle, provided for the respective flow paths; and a plurality of second heaters heating a product solution generated by the PCR to a predetermined temperature so as to discharge the product solution to the outside of the arrayer, provided for the respective flow paths.

The first and the second heaters may be placed inside the substrate. The substrate may be composed of a heat conductive material.

The first heater may uniformly heat the entire PCR solution. The second heater may heat the product solution adjacent thereto in order to generate bubbles. For this, the second heater may be disposed closer to the flow path compared to the first heater.

According to another aspect of the present invention, there is provided an inkjet-type DNA arrayer including: a substrate having a plurality of manifolds storing a PCR solution and a plurality of nozzles through which a product solution generated by the PCR is discharged; a plurality of first heaters heating the PCR solution according to the PCR temperature cycle; and a plurality of second heaters heating the product solution to a predetermined temperature in order to discharge the product solution through the nozzles.

The first and the second heaters may be placed inside the substrate. The substrate may be composed of silicon or metal.

The substrate may further have a plurality of chambers communicating with the nozzles and a plurality of channels connecting the chambers with the manifolds.

The first heater may uniformly heat the entire PCR solution. The second heater may heat the product solution in the chamber to generate bubbles. For this, the second heater may be disposed closer to the chamber compared to the first heater. The second heater may be formed so as to surround the nozzle.

The first and the second heaters may be composed of at least one material selected from the group consisting of polysilicon, tantalum-aluminum alloy, tantalum nitride, titanium nitride and tungsten silicide.

According to another aspect of the present invention, there is provided a method of manufacturing DNA microarrays using the inkjet-type DNA arrayer, the method including: performing a PCR by heating a PCR solution with a first heater according to the PCR temperature cycle; and forming a spot array by heating a product solution generated by the PCR with a second heater in order to discharge the product solution onto a solid substrate.

### BRIEF DESCRIPTION OF THE DRAWINGS

The above and other features and advantages of the present invention will become more apparent by describing in detail exemplary embodiments thereof with reference to the attached drawings in which:

FIG. 1 is a schematic cross-sectional view of a conventional micro PCR apparatus;

FIG. 2 is a graph illustrating the temperature distribution of a PCR solution with respect to time when heating the PCR solution with a heater in the conventional micro PCR apparatus illustrated in FIG. 1;

FIG. 3 is a schematic plan view of an inkjet-type DNA arrayer capable of performing a PCR according to an embodiment of the present invention;

FIG. 4 is a cross-sectional view of the inkjet-type DNA arrayer of FIG. 3, taken along the line IV-IV′;

FIG. 5 is a plan view of FIG. 4;

FIGS. 6A though 6D are the simulation results showing the temperature distribution of the PCR solution at 1 ms, 0.01 s, 0.02 s, and 0.04 s, respectively, when heating the PCR solution with a first heater in the inkjet-type DNA arrayer according to an embodiment of the present invention; and

FIG. 7 is the simulation results showing the temperature distribution of the PCR solution with respect to time when heating the PCR solution with the first heater in the inkjet-type DNA arrayer according to an embodiment of the present invention.

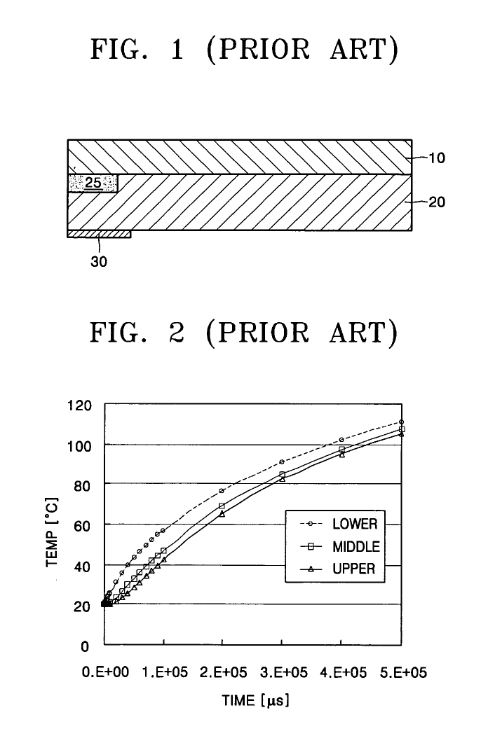

### DETAILED DESCRIPTION OF THE INVENTION

The present invention will now be described more fully with reference to the accompanying drawings, in which embodiments of the invention are shown. In the drawings, like reference numbers refer to like elements throughout.

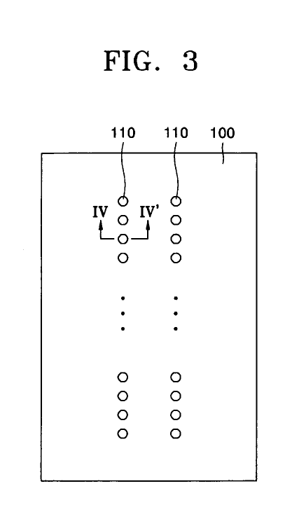

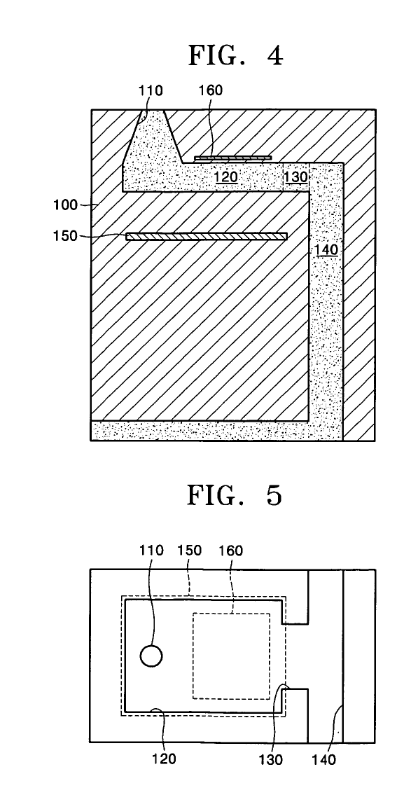

FIG. 3 is a schematic plan view of an inkjet-type DNA arrayer that is capable of performing a PCR according to an embodiment of the present invention. FIG. 4 is a cross-sectional view of the inkjet-type DNA arrayer of FIG. 3, taken along the line IV-IV′ and FIG. 5 is a plan view of FIG. 4.

Referring to FIG. 3, a plurality of nozzles 110 are arranged in two parallel rows on a substrate 100. In the present embodiment, the nozzles 110 can also be arranged in one row or in three rows or more on the substrate 100 unlike in FIG. 3.

Referring to FIGS. 4 and 5, an inkjet-type DNA arrayer according to an embodiment of the present invention includes a substrate 100 having a plurality of flow paths, and a plurality of first and second heaters 150, 160 provided inside the substrate 100, corresponding to the respective flow paths.

The substrate 100 is preferably composed of a high heat conductive material such as silicon or metal. Each flow path includes a manifold 140 formed on the rear of the substrate 100, a nozzle 110 formed on a surface of the substrate 100, a chamber 120 communicating with the nozzle 100, and a channel 130 connecting the chamber 120 and the manifold 140. A polymerase chain reaction (PCR) solution, including a template DNA, is filled in the flow path. The PCR solution for performing a thermal cycling reaction that proliferates DNA includes dNTP, ions, such as Mg²⁺, primers, polymerases, and the like.

The manifold 140 stores the PCR solution injected from an external source and is connected to a solution inlet formed in a glass substrate (not shown) which is attached to the rear of the substrate 100. Thus, the PCR solution is supplied from the manifold 140 to the chamber 120 and the nozzle 110 via the channel 130.

In order to perform the PCR, the first heater 150 heats the PCR solution filled in the flow path according to the PCR temperature cycle. Specifically, in the PCR, a thermal cycling process, including denaturation, annealing, and polymerization, is repeated, wherein the first heater 150 repeatedly heats the PCR solution according to a predetermined PCR temperature cycle (to about 90° C. or higher for denaturation, about 50° C. for annealing, and about 72° C. for polymerization).

The first heater 150 is provided directly below the chamber 120 and inside the substrate 100 and is preferably located away from the flow path so as to uniformly heat the entire PCR solution filled in the flow path. The first heater 150 may be composed of a heating resistor such as polysilicon, tantalum-aluminum alloy, tantalum nitride, titanium nitride, tungsten silicide, and the like.

The second heater 160 heats a product solution generated by the PCR to a predetermined temperature in order to discharge the product solution to outside of the arrayer. Specifically, if a PCR takes place in the PCR solution due to the first heater 150, a product solution, including proliferated DNA, is generated as a product of the reaction in the flow path. The second heater 160 heats the product solution in the chamber 120 to a predetermined temperature, for example, to 200° C. or higher in order to generate bubbles which discharge the product solution through the nozzle 110.

The second heater 160 is provided directly above the chamber 120 and inside the substrate 100 and is preferably located closer to the chamber 120 compared to the first heater 150. Thus, the second heater 160 heats the product solution adjacent thereto in order to generate bubbles. The second heater 160 may be composed of a material such as the heating resistor composing the first heater 150 as described above. Meanwhile, although the second heater 160 has a rectangular design in FIGS. 4 and 5, the second heater 160 can have various designs. For example, the second heater 160 can have a ring shape in order to surround the nozzle 110.

A method of manufacturing DNA microarrays using the inkjet-type DNA arrayer will now be described.

First, under the conditions that the PCR solution is filled in the flow path including the manifold 140, the channel 130, the chamber 120, and the nozzle 110, the PCR occurs by repeatedly heating the PCR solution with the first heater 150 according to a predetermined PCR temperature cycle. At this time, the entire PCR solution is uniformly heated in the flow path. Thus, a product solution generated by the PCR is filled in the flow path.

Then, the product solution contained in the chamber 120 is heated to a predetermined temperature by the second heater 160 in order to generate bubbles. The product solution around the nozzle 110 is discharged to a predetermined location on a solid substrate (not shown) by the expansive forces of the bubbles so as to form a DNA spot.

When this process is performed in each flow path in order to discharge the product solution through each nozzle 110, a predetermined DNA spot array is formed on the solid substrate, thereby resulting in a DNA microarray.

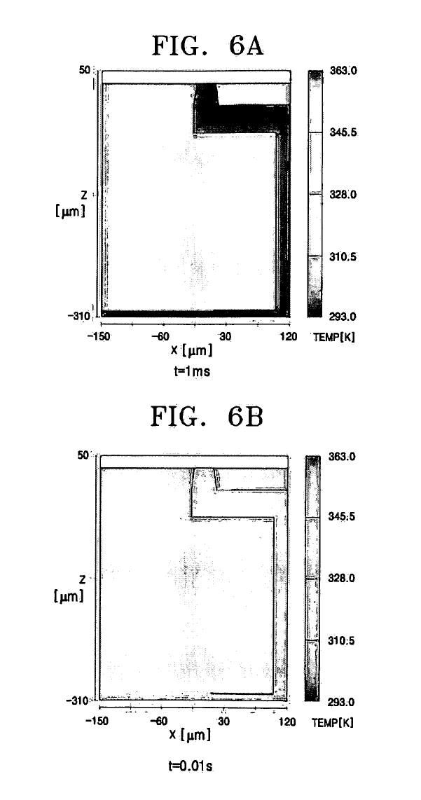

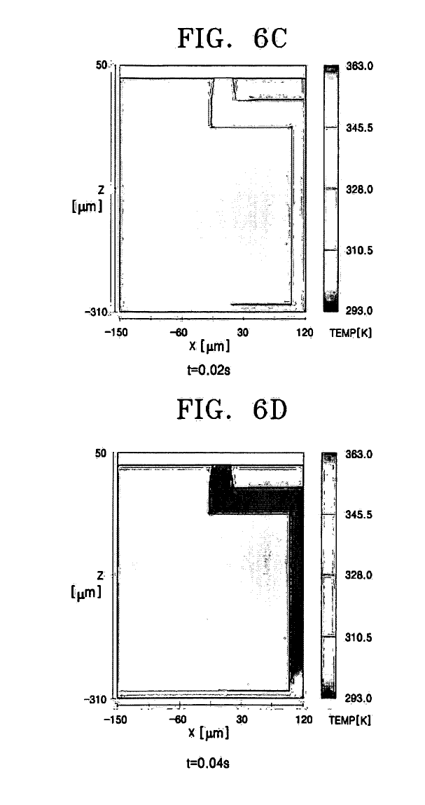

FIGS. 6A through 6D are the simulation results showing the temperature distribution of the PCR solution at 1 ms, 0.01 s, 0.02 s, and 0.04 s, respectively, when heating the PCR solution filled in the flow path with the first heater 150 in the inkjet-type DNA arrayer according to an embodiment of the present invention.

Referring to FIGS. 6A through 6D, the PCR solution filled in the flow path has a uniform temperature distribution. This indicates that heat generated from the first heater is uniformly transferred through the high heat conductive substrate to the entire PCR solution in the flow path.

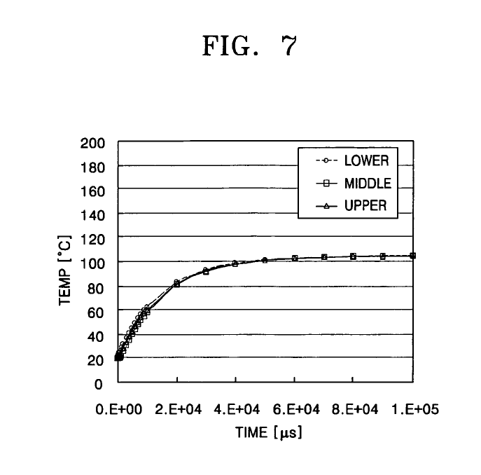

FIG. 7 shows the simulation results showing the temperature distribution of the PCR solution in the chamber 120 with respect to time when heating the PCR solution with the first heater 150 in the inkjet-type DNA arrayer according to an embodiment of the present invention. In FIG. 7, the height of the chamber 120 is 40 μm and the lower portion, the middle portion, and the upper portion represent points of 1 μm, 20 μm, and 39.7 μm, respectively, from the bottom of the chamber 120.

Referring to FIG. 7, the temperature distribution of the PCR solution contained in the chamber 120 is uniform at said portions. Thus, in the inkjet-type DNA arrayer according to an embodiment of the present invention, since the entire PCR solution is uniformly heated in the chamber 120, the reproducibility of the PCR proliferation results can be improved. Also, in the inkjet-type DNA arrayer according to an embodiment of the present invention, the PCR solution can heated to a desired temperature relatively quickly compared to the conventional micro PCR apparatus.

As described above, the inkjet-type DNA arrayer according to an embodiment of the present invention has the following effects.

First, the PCR for DNA proliferation and DNA spotting for manufacturing DNA microarrays can be simultaneously implemented.

Second, since the PCR solution can be heated to a desired temperature relatively quickly compared to the conventional micro PCR apparatus and the temperature distribution of the heated PCR solution is uniform, the reproducibility of PCR proliferation results is improved.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. A DNA arrayer comprising:
a substrate having a plurality of flow paths filled with a polymerase chain reaction (PCR) solution;
a plurality of first heaters heating the PCR solution according to a PCR temperature cycle, provided for the respective flow paths; and
a plurality of second heaters heating a product solution generated by the PCR to a predetermined temperature in order to discharge the product solution to the outside of the arrayer, provided for the respective flow paths.

2. The DNA arrayer of claim 1, wherein first and second heaters are placed in the inside of the substrate.

3. The DNA arrayer of claim 1, wherein the substrate is composed of a heat conductive material.

4. The DNA arrayer of claim 1, wherein the first heater uniformly heat the entire PCR solution.

5. The DNA arrayer of claim 1, wherein the second heater heats the product solution adjacent thereto in order to generate bubbles.

6. The DNA arrayer of claim 5, wherein the second heater is located closer to the flow path compared to the first heater.

7. A DNA arrayer comprising:
a substrate having a plurality of manifolds storing a PCR solution and a plurality of nozzles through which a product solution generated by the PCR is discharged;
a plurality of first heaters heating the PCR solution according to the PCR temperature cycle; and
a plurality of second heaters heating the product solution to a predetermined temperature in order to discharge the product solution through the nozzles.

8. The DNA arrayer of claim 7, wherein the first and the second heaters are located inside the substrate.

9. The DNA arrayer of claim 8, wherein the substrate is composed of a heat conductive material.

10. The DNA arrayer of claim 9, wherein the substrate is composed of silicon.

11. The DNA arrayer of claim 9, wherein the substrate is composed of a metal.

12. The DNA arrayer of claim 7, wherein the substrate further has a plurality of chambers communicating with the nozzles and a plurality of channels connecting the chambers with the manifolds.

13. The DNA arrayer of claim 12, wherein the first heater uniformly heats the entire PCR solution.

14. The DNA arrayer of claim 12, wherein the second heater heats the product solution in the chamber in order to generate bubbles.

15. The DNA arrayer of claim 14, wherein the second heater is located closer to the chamber compared to the first heater.

16. The DNA arrayer of claim 15, wherein the second heater is formed so as to surround the nozzle.

17. The DNA arrayer of claim 14, wherein the second heater heats the product solution to 200° C. or higher.

18. The DNA arrayer of claim 7, wherein the first and the second heaters are composed of at least one material selected from the group consisting of polysilicon, tantalum-aluminum alloy, tantalum nitride, titanium nitride and tungsten silicide.

19. A method of manufacturing DNA microarrays using a DNA arrayer including a substrate having a plurality of flow paths filled with a PCR solution and a plurality of first and second heaters provided for the respective flow paths, the method comprising:
performing a PCR by heating the PCR solution with the first heater according to the PCR temperature cycle; and
forming a spot array by heating a product solution generated by the PCR with the second heater in order to discharge the product solution onto a solid substrate.

20. The method of claim 19, wherein the first heater uniformly heats the PCR solution.

21. The method of claim 19, wherein the second heater heats the product solution adjacent thereto in order to generate bubbles.

22. The method of claim 21, wherein the second heater heats the product solution to 200° C. or higher.
